(19) United States
(12) Patent Application Publication
(10) Pub. No.: US 2025/0228806 A1
(43) Pub. Date: Jul. 17, 2025

(54) AMPK ACTIVATOR, MOTOR FUNCTION IMPROVING AGENT, MUSCULAR ENDURANCE IMPROVING AGENT, AND MUSCLE ATROPHY PREVENTING AGENT

(71) Applicant: MARUZEN PHARMACEUTICALS CO., LTD., Onomichi-shi, Hiroshima (JP)

(72) Inventor: Susumu YOSHINO, Fukuyama-shi (JP)

(21) Appl. No.: 18/701,698

(22) PCT Filed: Oct. 14, 2022
(86) PCT No.: PCT/JP2022/038465

§ 371 Date: Apr. 16, 2024

(30) Foreign Application Priority Data
Oct. 19, 2021 (JP) 2021-170940

(51) Int. Cl. A61K 31/192; A61K 9/00; A61P 21/00

(52) U.S. Cl. CPC A61K 31/192; A61K 9/0053; A61P 21/00

(57) ABSTRACT

A compound represented by Formula (I) below is used as an active ingredient of an AMPK activator, a motor function improving agent, a muscular endurance improving agent, and a muscle atrophy preventing agent according to the present invention. The compound represented by Formula (I) is compounded to an oral composition for AMPK activation purposes, an oral composition for motor function improvement purposes, an oral composition for muscular endurance improvement purposes, and an oral composition for muscle atrophy prevention purposes according to the present invention.

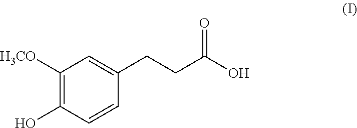 [SMILES: COc1cc(CCC(=O)O)ccc1O]

### TECHNICAL FIELD

The present invention relates to an AMPK activator, a motor function improving agent, a muscular endurance improving agent, and a muscle atrophy preventing agent that contain compounds derived from natural products as active ingredients. Furthermore, the present invention also relates to an oral composition for AMPK activation purposes, an oral composition for motor function improvement purposes, an oral composition for muscular endurance improvement purposes, an oral composition for muscle atrophy prevention purposes that are compounded with such compounds.

### BACKGROUND ART

Muscles are essential organs for all daily activities such as standing, walking, and maintaining posture, so maintaining the muscle functions and, if possible, improving them, is very important for living a healthy daily life. It is widely recognized that regular exercise is important for maintaining the muscle functions, but in adults, who tend to lack exercise, a decline in the muscle functions, such as a marked decrease in muscle mass, is observed, coupled with the effects of aging. In particular, elderly people generally have less muscle mass and are reduced in exercise ability, and it may be desirable to suppress the decline in muscle mass in order to live a safe life. In addition, when long-term bed rest is required after surgery or due to illness treatment, etc., muscle mass may decrease, preventing early return to daily life after treatment, and it is therefore desirable to suppress the decrease in muscle mass.

On the other hand, people who prefer exercise (e.g., athletes, sports enthusiasts, etc.) may desire to increase muscle mass in order to improve their athletic ability. Furthermore, in order to improve athletic ability, it is important to increase muscle mass and improve muscular endurance.

A possible way to improve muscular endurance is to improve the energy metabolism function of muscle cells. It is believed that a signal pathway mediated by AMPK (5′-AMP-activated protein kinase) is important for energy metabolism in muscle cells (Non-Patent Documents 1 and 2). When a living body is state of energy deficiency, the intracellular AMP concentration increases and AMPK is activated. Activation of AMPK in muscle cells promotes the expression of PGC-1α (peroxisome proliferator activated receptor γ coactivator 1α, Gene Symbol: Ppargc1a), which is a transcriptional coactivator. PGC-1α is involved in mitochondrial biogenesis, and promoting the expression of PGC-1α is expected to enhance the energy metabolism and endurance through mitochondrial biogenesis. In addition, activation of AMPK promotes the expression of GLUT4 (glucose transporter 4, Gene Symbol: Slc2a4), which is a glucose transporter, in an insulin-independent manner, induces the translocation of GLUT4 onto the cell membrane, and promotes the intracellular transport of glucose. Promotion of the expression of GLUT4 and its translocation onto the cell membrane promote the intracellular transport of glucose, and the blood sugar levels are reduced. It is thus believed that promotion of the expression of GLUT4 and its translocation onto the cell membrane are involved in the prevention of hyperglycemia, diabetes, diabetes-related diseases (e.g., obesity, hyperlipidemia, hypercholesterolemia, dyslipidemia, hypertension, fatty liver, metabolic syndrome, and arteriosclerosis) or diabetic complications (e.g., retina disease, nephropathy, neuropathy, cataract, and ketosis).

On the other hand, it is known that the increase in muscle mass (or suppression of its decrease) is associated with a signal pathway mediated by Akt (also known as protein kinase B). Akt is a serine/threonine kinase that is activated by growth hormone, exercise stimulation, etc. When Akt is activated, it induces the activation of mTOR (mammalian target of rapamycin) to promote protein synthesis, suppresses the expression of MuRF-1 (Gene Symbol: Trim63), MAFbx/Atrogin-1 (Gene Symbol: Fbxo32), etc., which are known as muscle atrophy-related genes, and suppresses muscle catabolism (degradation) (Non-Patent Documents 3 and 4).

In recent years, it has been discovered that cytokines are secreted from skeletal muscle, and the concept of myokines has been proposed. The first myokine discovered is interleukin-6 (IL-6). IL-6 has been known as an inflammatory cytokine, but it is believed that the secretion of IL-6 from skeletal muscle is correlated with exercise duration and muscle mass and is involved in muscle hypertrophy and maintenance of metabolic function (Non-Patent Document 5).

What is known as having an AMPK activating action is 5-amino-4-imidazolecarboxamide ribonucleoside (AICAR) (Non-Patent Document 6). In addition, polymeric polyphenols extracted from fermented tea (Patent Document 1), black ginger extract (Patent Document 2), etc. have been reported to have a muscular endurance improving action.

On the other hand, the use of growth hormones and steroids is known as a method of increasing muscle mass, but these have the problem of side effects. In addition, ornithine or its salts (Patent Document 3), Kaempferia parviflora extract and flavone-based compounds isolated therefrom (Patent Document 4), etc. have been reported to have a muscle atrophy preventing action or a muscle mass increasing action.

### PRIOR ART DOCUMENTS

### Patent Documents

[Patent Document 1] JP2010-037323A
[Patent Document 2] JP2016-008180A
[Patent Document 3] WO2007/077995
[Patent Document 4] WO2013/172681

### Non-Patent Documents

[Non-Patent Document 1] Med. Sci. Sports Exerc., 2006, vol. 38, pp. 1945-1949
[Non-Patent Document 2] Diabetes, 2012, vol. 55, No. 5, pp. 306-308
[Non-Patent Document 3] Biochemistry, 2014, vol. 86, No. 3, pp. 367-371
[Non-Patent document 4] FEBS J, 2013, vol. 280, pp. 4294-4314
[Non-Patent Document 5] FEBS J., 2013, vol. 280, pp. 4131-4148
[Non-Patent Document 6] FEBS Lett., 1994, vol. 353, pp. 33-36

### SUMMARY OF THE INVENTION

### Problems to be Solved by the Invention

An object of the present invention is to find, from among compounds derived from natural products, those having excellent actions in an AMPK activating action, a motor function improving action, a muscular endurance improving action, and a muscle atrophy preventing action and provide an AMPK activator, a motor function improving agent, a muscular endurance improving agent, and a muscle atrophy preventing agent that contain the above compounds as the active ingredients.

Another object of the present invention is to provide oral compositions for AMPK purposes, motor function improvement purposes, muscular endurance improvement purposes, or muscle atrophy prevention purposes that are compounded with compounds derived from natural products having excellent actions in an AMPK activating action, a motor function improving action, a muscular endurance improving action, and a muscle atrophy preventing action.

### Means for Solving the Problems

To solve the above problems, the AMPK activator, motor function improving agent, muscular endurance improving agent, and muscle atrophy preventing agent of the present invention are characterized by comprising a compound represented by Formula (I) below as an active ingredient. Additionally or alternatively, the oral composition for AMPK activation purposes, oral composition for motor function improvement purposes, oral composition for muscular endurance improvement purposes, and oral composition for muscle atrophy prevention purposes of the present invention are characterized by compounding the compound represented by Formula (I) below.

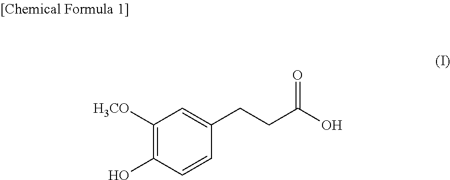 [SMILES: COc1cc(CCC(=O)O)ccc1O]

### Advantageous Effect of the Invention

According to the present invention, by using the compound represented by the above Formula (I) as an active ingredient, it is possible to provide the AMPK activator, motor function improving agent, muscular endurance improving and muscle atrophy preventing agent that have agent, excellent actions and effects. Furthermore, by compounding the compound represented by the above Formula (I), it is possible to provide the oral compositions suitable for AMPK activation purposes, motor function improvement purposes, muscular endurance improvement purposes, and muscle atrophy prevention purposes.

### EMBODIMENTS FOR CARRYING OUT THE INVENTION

One or more embodiments of the present invention will be described below.

The AMPK activator, motor function improving agent, muscular endurance improving agent, and muscle atrophy preventing agent of the present embodiment comprise a compound represented by Formula (I) below as an active ingredient. Additionally or alternatively, the oral composition for AMPK activation purposes, oral composition for motor function improvement purposes, oral composition for muscular endurance improvement purposes, and oral composition for muscle atrophy prevention purposes of the present embodiment are obtained by compounding the compound represented by Formula (I) below.

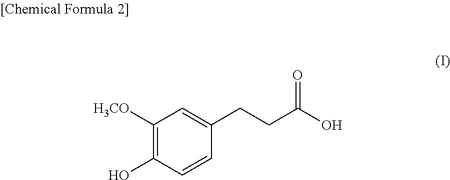 [SMILES: COc1cc(CCC(=O)O)ccc1O]

The compound represented by the above Formula (I) is a cinnamic acid derivative also called 3-(4-hydroxy-3-methoxyphenyl) propionic acid. In the present specification, the compound represented by the above Formula (I) may be referred to as a compound (I), hereinafter.

The compound (I) can be produced, for example, by isolation/purification from plant extracts containing the compound (I). In this case, such plant extracts containing the compound (I) can be obtained by a method that is commonly used for extraction from plants. Examples of plants containing the compound (I) include rice, barley, wheat, soybeans, adzuki beans, and corn.

The compound (I) can also be produced, for example, through fermenting 3-(4-hydroxy-3-methoxyphenyl) propenoic acid or its derivatives or a composition containing them (e.g., a crushed material or extract of a plant, or the like) with a microorganism having phenolic acid reductase to convert the 3-(4-hydroxy-3-methoxyphenyl) propenoic acid into the compound (I) and then extracting/isolating/purifying the resulting fermented product. Examples of compositions containing the 3-(4-hydroxy-3-methoxyphenyl) propenoic acid include crushed products and extracts of plants such as coffee, wheat, corn, tomato, mate, mugwort, and burdock. The 3-(4-hydroxy-3-methoxyphenyl) propenoic acid is a constituent of lignin in woody plants and herbaceous plants, and lignin or a composition containing it may therefore be used as a fermentation raw material. On the other hand, examples of microorganisms having phenolic acid reductase include lactic acid bacteria such as Lactobacillus plantarum, Lactobacillus fermentum, Lactobacillus gasseri, Lactobacillus johnsonii, Lactobacillus crispatus, Lactobacillus acidophilus, Lactobacillus amylovorus, Lactobacillus delbrueckii, Lactobacillus buchneri, Lactobacillus kefiranofaciens Lactobacillus gallinarum and Enterococcus faecalis.

The method of extracting/isolating/purifying the compound (I) from the above plants or fermented products is not particularly limited, and can be carried out according to an ordinary method. For example, the extraction process may be performed through drying the above plants or fermented products as extraction raw materials, then as they are or pulverizing them using a coarse pulverizer, and subjecting them to extraction with an extraction solvent. Drying may be performed in the sun or using a commonly used dryer. They may also be used as extraction raw materials after being subjected to preprocessing such as degreasing with a nonpolar solvent such as hexane. By performing preprocessing such as degreasing, the extraction process using a polar solvent can be performed efficiently.

It is preferred to use a polar solvent as the extraction solvent, and examples of the polar solvent include water, hydrophilic organic solvents, etc., which may be used alone or in combination of two or more at room temperature or a temperature equal to or lower than the boiling point of the solvent.

Examples of water that can be used as the extraction solvent include pure water, tap water, well water, mineral spring water, mineral water, hot spring water, spring water, fresh water, etc. and those that have been subjected to various processes. Examples of the processes applied to water purification, include heating, sterilization, filtration, ion exchange, osmotic pressure adjustment, and buffering. Therefore, examples of water that can be used as the extraction solvent in the present embodiment also include purified water, hot water, ion exchange water, physiological saline, phosphate buffer solution, and phosphate buffered saline.

Examples of hydrophilic organic solvents that can be used as extraction solvents include lower aliphatic alcohols whose carbon number is 1 to 5, such as methanol, ethanol, propyl alcohol, and isopropyl alcohol; polyhydric alcohols whose carbon number is 2 to 5, such as 1,3-butylene glycol, propylene glycol, and glycerin; and lower aliphatic ketones whose carbon number is 3 to 5, such as acetone and methyl ethyl ketone.

When a mixed solution of two or more polar solvents is used as the extraction solvent, the mixing ratio is arbitrary and can be adjusted as appropriate. For example, when a mixed solution of water and a hydrophilic organic solvent is used as the extraction solvent, they can be mixed and used at any ratio, that is, more than 0:100 and less than 100:0 (volume ratio, here and hereinafter), and the mixing ratio can be adjusted as appropriate.

For example, when a mixed solution of water and lower aliphatic alcohol is used as the extraction solvent, the mixing ratio (volume ratio) of water and lower aliphatic alcohol can be 9:1 or more in an embodiment or 7:3 or more in another embodiment, or the mixing ratio of water and lower aliphatic alcohol can be 1:9 or less in an embodiment or 2:8 or less in another embodiment. Additionally or alternatively, when a mixed solution of water and polyhydric alcohol is used, the mixing ratio of water and polyhydric alcohol can be 8:2 or more in an embodiment or 1:9 or less in another embodiment, and when a mixed solution of water and lower aliphatic ketone is used, the mixing ratio of water and lower aliphatic ketone can be 9:1 or more in an embodiment or 2:8 or less in another embodiment.

The extraction process is not particularly limited, provided that the soluble components contained in the extraction raw material can be eluted into the extraction solvent, and can be performed according to an ordinary method. For example, the extraction liquid can be obtained through immersing the extraction raw material in an extraction solvent of 5 to 15 times the amount of the extraction raw material (mass ratio), extracting the soluble components at an ordinary temperature or under reflux heating, and then removing the extraction residue by filtration. When the solvent is distilled off from the resulting extraction liquid, a paste-like concentrate is obtained, and when this concentrate is further dried, a dry product is obtained.

The method of isolating/purifying the compound (I) from the extraction liquid obtained as above, the concentrate of the extraction liquid, or the dried product of the extraction liquid is not particularly limited, and can be carried out by an ordinary method. Examples of the method include a method of dissolving the extract in a developing solvent and subjecting it to column chromatography using a porous material such as silica gel or alumina, a porous resin such as styrene-divinylbenzene copolymer or polymethacrylate, or the like to collect a fraction containing the compound (I). In this case, the developing solvent may be appropriately selected depending on the stationary phase used. For example, when extracts are separated by normal phase chromatography using silica gel as the stationary phase, examples of the developing solvent include chloroform:methanol=95:5. Furthermore, the fraction containing the compound (I) obtained by column chromatography may be purified using any organic compound purification means such as reverse phase silica gel chromatography using ODS, recrystallization, liquid-liquid countercurrent extraction, or column chromatography using ion exchange resin.

### <AMPK Activator, Motor Function Improving Agent, Muscular Endurance Improving Agent, and Muscle Atrophy Preventing Agent>

The compound (I) obtained in the above manner has excellent actions in an AMPK activating action, a motor function improving action, a muscular endurance improving action, and a muscle atrophy preventing action and can therefore be used as the active ingredients of an AMPK activator, a motor function improving agent, a muscular endurance improving agent, and a muscle atrophy preventing agent.

In other words, the compound (I) can be used for producing an AMPK activator, a motor function improving agent, a muscular endurance improving agent, and a muscle atrophy preventing agent.

The AMPK activator, motor function improving agent, muscular endurance improving agent, and muscle atrophy preventing agent of the present embodiment can be used in a wide range of applications such as pharmaceuticals, quasi-drugs, and cosmetics.

Here, the motor function improving action possessed by the compound (I) may be exerted, for example, based on one or more actions selected from the group consisting of the previously described AMPK activating action, muscular endurance improving action, and muscle atrophy preventing action as well as a PGC-1α mRNA expression promoting action, a GLUT4 mRNA expression promoting action, an IL-6 mRNA expression promoting action, a muscle mass increasing action, an MuRF-1 mRNA expression suppressing action, an Atrogin-1 mRNA expression suppressing action, and an Akt activating action. However, the motor function improving action possessed by the compound (I) is not limited to the motor function improving action exerted based on the above action or actions.

Here, the muscular endurance improving action possessed by the compound (I) may be exerted, for example, based on one or more actions selected from the group consisting of the AMPK activating action, PGC-1α mRNA expression promoting action, GLUT4 mRNA expression promoting action, and IL-6 mRNA expression promoting action. However, the muscular endurance improving action possessed by the compound (I) is not limited to the muscular endurance improving action exerted based on the above action or actions.

The muscular endurance improving agent of the present embodiment can be used for PGC-1α mRNA expression promotion purposes, GLUT4 mRNA expression promotion purposes, or IL-6 mRNA expression promotion purposes, respectively, using the PGC-1α mRNA expression promoting action, GLUT4 mRNA expression promoting action, or mRNA expression IL-6 promoting action of the compound (I).

In other words, the compound (I) can also be used as the active ingredients of a PGC-1α mRNA expression promoter, a GLUT4 mRNA expression promoter, or an IL-6 mRNA expression promoter.

Here, the muscle atrophy preventing action possessed by the compound (I) may be exerted, for example, based on one or more actions selected from the group consisting of a muscle mass increasing action, an MuRF-1 mRNA expression suppressing action, an Atrogin-1 mRNA expression suppressing action, and an Akt activating action. However, the muscle atrophy preventing action possessed by the compound (I) is not limited to the muscle atrophy preventing action exerted based on the above action or actions.

The muscle atrophy preventing agent of the present embodiment can be used for muscle mass increasing purposes, MuRF-1 mRNA expression suppressing purposes, Atrogin-1 mRNA expression suppressing purposes, or Akt activating purposes, respectively, using the muscle mass increasing action, MuRF-1 mRNA expression suppressing action, Atrogin-1 mRNA expression suppressing action, and Akt activating action of the compound (I).

In other words, the compound (I) can also be used as the active ingredients of a muscle mass increasing agent, an MuRF-1 mRNA expression suppressing agent, an Atrogin-1 mRNA expression suppressing agent, or an Akt activator.

Compositions containing the compound (I) as substitute for the compound (I) isolated may be used as the active ingredients of the AMPK activator, motor function improving agent, muscular endurance improving agent, and muscle atrophy preventing agent according to the present embodiment. Here, the “compositions containing the compound (I)” in the present embodiment include extracts obtained from plants containing the compound (I) as the extraction raw materials, fermented products containing the compound (I), and extracts obtained using the fermented products as the extraction raw materials. The “extracts” include an extraction liquid obtained by an extraction process, a diluted or concentrated liquid of the extraction liquid, or a dried product obtained by drying the extraction liquid.

When the compositions containing the compound (I) are used as the active ingredients of the AMPK activator, motor function improving agent, muscular endurance improving agent, and muscle atrophy preventing agent according to the present embodiment, the content of the compound (I) in the compositions is preferably 0.1 mass % or more, more preferably 5 mass % or more, and particularly preferably 50 mass % or more. By using the compound (I) with increased purity as the active ingredients, the AMPK activator, motor function improving agent, muscular endurance improving agent, and muscle atrophy preventing agent can be obtained with even more excellent actions and effects.

The AMPK activator, motor function improving agent, muscular endurance improving agent, and muscle atrophy preventing agent according to the present embodiment may consist only of the compound (I) or compositions containing the compound (I) or may also be obtaining by formulating the compound (I) or compositions containing the compound (I).

The AMPK activator, motor function improving agent, muscular endurance improving agent, and muscle atrophy preventing agent according to the present embodiment can be formulated into any dosage form such as powder, granules, tablets, or liquid according to an ordinary method using a pharmaceutically acceptable carrier such as dextrin or cyclodextrin or any other auxiliary agent. In this case, examples of usable auxiliary agents include an excipient, a binder, a disintegrant, a lubricant, a stabilizer, and a flavoring/odor ameliorating agent. The AMPK activator, motor function improving agent, muscular endurance improving agent, and muscle atrophy preventing agent can be used by being compounded with other compositions (e.g., skin cosmetics, hair cosmetics, etc.) or can also be used as ointments, liquids for external use, adhesive skin patches, etc.

When the AMPK activator, motor function improving agent, muscular endurance improving agent, and muscle atrophy preventing agent according to the present embodiment is formulated, the content of the compound (I) or compositions containing the compound (I) is not particularly limited and can be set as appropriate depending on the purpose.

The AMPK activator, motor function improving agent, muscular endurance improving agent, and muscle atrophy preventing agent according to the present embodiment can be used as active ingredients by compounding other natural extracts or the like having an AMPK activating action, a motor function improving action, a muscular endurance improving action, and a muscle atrophy preventing action as necessary together with the compound (I) or compositions containing the compound (I).

Examples of methods for administering the AMPK activator, motor function improving agent, muscular endurance improving agent, and muscle atrophy preventing agent according to the present embodiment include oral administration and transdermal administration. Depending on the type of disease, a method suitable for its prevention or treatment or the like may be appropriately selected. The dosage amount of the AMPK activator, motor function improving agent, muscular endurance improving agent, and muscle atrophy preventing agent according to the present embodiment may be increased or decreased as appropriate depending on the type of disease, severity, individual differences between patients, administration method, administration period, etc.

The AMPK activator of the present embodiment can activate the AMPK through activating action possessed by the compound (I), which is an active ingredient, thereby to improve the energy metabolism in muscles. Fortunately, however, the AMPK activator of the present embodiment can be used not only for these applications but also for all applications that are meaningful in exerting the AMPK activating action.

For example, the AMPK activator of the present embodiment can activate sugar metabolism and lipid metabolism through its AMPK activating action, and can therefore be used to prevent, treat, or ameliorate diseases caused by abnormal sugar metabolism, such as diabetes, diabetes-related diseases, and diabetic complications; and diseases caused by abnormal lipid metabolism, such as hyperlipidemia, hypercholesterolemia, obesity, fatty liver, and arteriosclerosis.

The motor function improving agent of the present embodiment can improve the muscular endurance and suppress the muscle atrophy (or increase the muscle mass) thereby to improve the muscle functions and motor functions through the motor function improving action possessed by the compound (I), which is an active ingredient, for example, through one or more actions selected from the group consisting of an AMPK activating action, a muscular endurance improving action, a muscle atrophy preventing action, a PGC-1 mRNA expression promoting action, a GLUT4 mRNA expression promoting action, an IL-6 mRNA expression promoting action, a muscle mass increasing action, an MuRF-1 mRNA expression suppressing action, an Atrogin-1 mRNA expression suppressing action, and an Akt activating action. Fortunately, however, the motor function improving agent of the present embodiment can be used not only for these applications but also for all applications that are meaningful in exerting the AMPK activating action, muscular endurance improving action, muscle atrophy preventing action, PGC-1α mRNA expression promoting action, GLUT4 mRNA expression promoting action, IL-6 mRNA expression promoting action, muscle mass increasing action, MuRF-1 mRNA expression suppressing action, Atrogin-1 mRNA expression suppressing action, or Akt activating action.

The muscular endurance improving agent of the present embodiment can improve the energy metabolism in muscles and the muscular endurance through the muscular endurance improving action possessed by the compound (I), which is an active ingredient, for example, through one or more actions selected from the group consisting of an AMPK activating action, a PGC-1α mRNA expression promoting action, a GLUT4 mRNA expression promoting action, and an IL-6 mRNA expression promoting action. Fortunately, however, the muscular endurance improving agent of the present embodiment can be used not only for these applications but also for all applications that are meaningful in exerting the muscular endurance improving action, AMPK activating action, PGC-1α mRNA expression promoting action, GLUT4 mRNA expression promoting action, or IL-6 mRNA expression promoting action.

The muscle atrophy preventing agent of the present embodiment can suppress the muscle atrophy through the muscle atrophy preventing action possessed by the compound (I), which is an active ingredient, for example, through one or more actions selected from the group consisting of a muscle mass increasing an action, MuRF-1 mRNA expression suppressing action, an Atrogin-1 mRNA expression suppressing action, and an Akt activating action. Applications for suppressing muscle atrophy include: prevention, treatment, or amelioration of muscular atrophy-related diseases such as muscular atrophy, sacopenia, atony, muscular dystrophy, muscle degeneration, and myasthenia; decline-suppression, maintenance, or improvement of exercise capacity; decline-suppression, maintenance, or improvement of muscle strength; and improvement of basal metabolic rate. Fortunately, however, the muscle atrophy preventing agent of the present embodiment can be used not only for these applications but also for all applications that are meaningful in exerting the muscle atrophy preventing action, muscle mass increasing action, MuRF-1 mRNA expression suppressing action, Atrogin-1 mRNA expression suppressing action, or Akt activating action.

The AMPK activator, motor function improving agent, muscular endurance improving agent, and muscle atrophy preventing agent of the present embodiment have excellent actions in the AMPK activating action, motor function improving action, muscular endurance improving action, and muscle atrophy preventing action, respectively, and can therefore be suitably used as reagents for research on the mechanisms of these actions.

### <Oral Composition for AMPK Activation Purposes, Oral Composition for Motor Function Improvement Purposes, Oral Composition for Muscular Endurance Improvement Purposes, and Oral Composition for Muscle Atrophy Prevention Purposes>

The compound (I) has excellent actions in the AMPK activating action, motor function improving action, muscular endurance improving action, and muscle atrophy preventing action, and is therefore suitable for being compounded in oral compositions. In this case, the compound (I) or compositions containing the compound (I) may be compounded as they are, or the AMPK activator, motor function improving agent, muscular endurance improving agent, or muscle atrophy preventing agent formulated from the compound (I) may also be compounded.

By compounding the oral composition with the compound (I) or compositions containing the compound (I) or with the AMPK activator, motor function improving agent, muscular endurance improving agent, or muscle atrophy preventing agent formulated from the compound (I) or from compositions containing the compound (I), the oral composition can be suitable for AMPK activation purposes, motor function improvement purposes, endurance muscular improvement purposes, or muscle atrophy prevention purposes. The above actions are suitable because they are likely to exhibit their actions and effects when given to the oral composition.

Here, the oral composition refers to a composition that has little risk of harming human health and is ingested orally or through gastrointestinal administration in normal social life, and is not restricted by administrative classifications such as foods, pharmaceuticals, and quasi-drugs. Therefore, the “oral composition” in the present embodiment refers to general foods, feeds, health foods, foods with health claims (foods for specified health uses, foods with nutrient function claims, foods and drinks with functional claims), quasi-drugs, pharmaceuticals, etc. that are ingested orally, and includes a wide variety of products. The oral composition according to the present embodiment is preferably an oral composition that can display the favorable actions possessed by the compound (I) on the oral composition or its packaging, and particularly preferably any of foods with health claims (foods for specified health uses, foods and drinks with functional claims, foods with nutrient function claims), quasi-drugs, or pharmaceuticals.

When compounding the oral composition with the compound (I) or compositions containing the compound (I) or with the AMPK activator, motor function improving agent, muscular endurance improving agent, or muscle atrophy preventing agent formulated from the compound (I) or from compositions containing the compound (I), the compounding amount of active ingredients therein can be changed as appropriate in consideration of the purpose of use, symptoms, gender, etc., but considering the daily intake of oral compositions as the additives, the daily intake of the compound (I) for adults is preferably about 1 to 1000 mg. When the oral composition to be added is in the form of granules, tablets, or capsules, the compounding amount of the compound (I) or compositions containing the compound (I) or the compounding amount of the AMPK activator, motor function improving agent, muscular endurance improving agent, or muscle atrophy preventing agent formulated from the compound (I) or from compositions containing the compound (I) is usually 0.1 to 100 mass % and preferably 5 to 100 mass % with respect to the oral composition to be added.

The oral composition of the present embodiment may be one in which the compound (I) is compounded into any oral composition that does not interfere with the activity of the compound (I), or may also be a nutritional supplements containing the compound (I) as the main component.

When producing the oral composition of the present embodiment, the oral composition can be formed in any shape by adding arbitrary auxiliary agents, for example, sugars such as dextrin and starch; proteins such as gelatin, soybean protein, and corn protein; amino acids such as alanine, glutamine, and isoleucine; polysaccharides such as cellulose and gum arabic; oils and fats such as soybean oil and medium-chain fatty acid triglyceride; and the like.

Oral compositions that can be compounded with the compound (I) are not particularly limited, but specific examples include beverages such as soft drinks, carbonated drinks, nutritional drinks, fruit drinks, and lactic acid drinks (including concentrated undiluted solutions of these drinks and powders for preparation); frozen desserts such as ice cream, ice sherbet, and shaved ice; noodles such as buckwheat noodle, wheat noodle, bean thread noodle, dumpling skin, Chinese dumpling skin, Chinese noodle, and instant noodle; sweets such as lollipop, chewing gum, candy, gum, chocolate, compressed tablet candy, snacks, biscuits, jellies, jams, creams, and baked confectionery; fishery/livestock processed foods such as fish minced and steamed, ham, and sausage; dairy products such as processed milk and fermented milk; fats and oils and their processed foods such as salad oil, fritter oil, margarine, mayonnaise, shortening, whipped cream, and dressings; seasonings such as sauces and mop sauces; soups, stews, salads, side dishes, and pickles; and various other forms of health/nutritional supplements; tablets, capsules, drinks, etc. When the compound (I) is compounded in these oral compositions, commonly used auxiliary raw materials and additives can be used in combination.

The AMPK activator, motor function improving agent, muscular endurance improving agent, muscle atrophy preventing agent, oral composition for AMPK activation purposes, oral composition for motor function improvement purposes, oral composition for muscular endurance improvement purposes, and oral composition for muscle atrophy prevention purposes of the present embodiment are suitably applied to humans, but can also be applied to animals other than humans (e.g., mice, rats, hamsters, dogs, cats, cows, pigs, monkeys, etc.), provided that the respective actions and effects are achieved.

### EXAMPLES

Hereinafter, the present invention will be specifically described with reference to testing examples, compounding examples, etc., but the present invention is not limited to the following examples. In these testing examples, the compound (I) (available from Tokyo Chemical Industry Co., Ltd., 3-(4-hydroxy-3-methoxyphenyl) propionic acid, Sample 1) was used as a test sample.

### <Testing Example 1> Muscle Atrophy Preventing Action Test

Using the compound (I) (Sample 1), the muscle atrophy preventing action on a rat muscle atrophy model was tested in the following manner.

Eight-week-old male Crlj:WI rats (purchased from Charles River Laboratories Japan, Inc.) were reared in an maintained environment of controlled temperature: 20.0-26.0° C., controlled humidity: 40.0-70.0% RH, light and dark for 12 hours each, and ventilation frequency: 12 times/hour. During the rearing period, solid feed (CRF-1, available from Oriental Yeast Co., Ltd.) was placed in a feeder and consumed ad libitum. However, during the hindlimb suspension rearing period described below, the rats were allowed to consume the feed by scattering it on the floor. For drinking water, tap water was available ad libitum using a water bottle. After a 5-day quarantine period and a 2-day acclimatization period, the rats were grouped with 8 rats per group as listed in Table 1 by random sampling so that the average weight and variance of each group were approximately equal.

TABLE 1

| | | Dosage of | |
|---|---|---|---|
| | | compound (I) | Number |
| Group | Name of group | (mg/kg) | of rats |
| 1 | Control group | 0 | 8 |
| 2 | Administration group | 125 | 8 |

The rats were put into a state in which the tails of the rats were attached to a rail on the ceiling with a rat tail suspension clip and the hindlimbs of the rats were off the floor (hindlimb suspension), and muscle atrophy was induced in this state by rearing the rats for 14 days after the grouping on the grouping day. During the period of rearing with suspended hindlimbs, the rats were able to move within the rearing cage using their forelimbs, and were able to freely consume water and feed.

In addition, the compound (I) was administered to the second group at a dose listed in Table 1. The compound (I) (Sample 1) was dissolved in water for injection and administered orally by force using a syringe to which a feeding needle was attached. The grouping day was taken as the first day (day 1 of administration), the administration was performed once a day for 14 days, and the day after the 14th day of administration (day 15 of administration) was the last day of the rearing period.

On the last day of the hindlimb suspension rearing (day 15 of administration), blood was collected from the abdominal aorta using a heparin-Na treated vacuum blood collection tube and a winged intravenous needle under isoflurane anesthesia. After blood collection, the rats were euthanized by exsanguination from the abdominal aorta, and the right and left gastrocnemius and soleus muscles were extracted. The extracted gastrocnemius and soleus muscles were weighed separately on the right and left sides (the values in Table 2 are the total values).

The results are listed in Table 2.

TABLE 2

| | | First group | Second group |
|---|---|---|---|
| | | (Control group) | (Administration group) |
| | | Dosage of compound (I) (mg/kg) | |
| | | 0 | 125 |
| Body weight | (g) | 310 | 309 |
| Soleus muscle | (mg) | 130.3 | 149.1 |
| (total of right and left) | | | |
| Gastrocnemius muscle | (mg) | 2.50 | 2.69 |
| (total of right and left) | | | |

As listed in Table 2, the weights of the soleus and gastrocnemius muscles were increased in the second group (administration group) as compared to the first group (control group). During this period, no difference was observed in the body weight between the first and second groups.

From the above, it was confirmed that the administration of the compound (I) increased the muscle mass.

### <Testing Example 2> Gene Expression Analysis Test in Muscle Atrophy Model

A gene expression analysis test using real-time PCR was conducted on the gastrocnemius muscle extracted in Testing Example 1 above.

The tissue of the gastrocnemius muscle extracted in Testing Example 1 above was fragmented, and total RNA was extracted using RNeasy® Lipid Tissue Mini Kit (available from Qiagen). The obtained total RNA was quantified using a spectrophotometer, and the total RNA was prepared to be 71.4 ng/μL.

Using 1000 ng of the total RNA as a template, a reverse transcription reaction was performed using SuperScript® VILO™ CDNA Synthesis Kit (available from Thermo Fisher Scientific). The reaction solution was 20 μL, and the operation was based on the manual attached to the above kit. A cDNA solution was obtained by performing the reverse transcription reaction (10 minutes at 25° C., 60 minutes at 42° C., and 5 minutes at 85° C.) using a thermal cycler (GeneAmp® PCR System 9700, available from Life Technologies).

Furthermore, CDNA was amplified using TaqMan® PreAmp Master Mix (available from Thermo Fisher Scientific). For amplification, all Taqman® Gene Expression Assays (available from Thermo Fisher Scientific) corresponding to the target gene and internal standard gene were mixed in one tube, diluted with TE buffer solution, and used. The assay IDs of Tagman® Gene Expression Assays corresponding to respective genes are Trim63 (assay ID: Rn 00590197_m1), Fbxo32 ID: Rn00591730_m1), Ppargc1a (assay ID: (assay Rn00580241 m1), Slc2a4 (assay ID: Rn00562597_m1), and I16 (assay ID: Rn01410330 m1). Gapdh (assay ID: Rn01775763_g1) was further used as an internal standard gene. The cDNA amplification reaction was performed using the above thermal cycler, and after 10 minutes at 95° C., 14 cycles of 15 seconds at 95° C.→4 minutes at 60° C. were performed.

Real-time PCR was performed using the obtained cDNA amplification solution as a template. Real-time PCR was performed using TaqMan® Universal Master Mix II, no UNG (available from Thermo Fisher Scientific). Assay mixes corresponding to the genes to be analyzed were prepared for respective genes using corresponding Taqman® Gene Expression Assays (the assay ID for each gene is described previously). The reaction conditions were: thermal mix (1 cycle of 2 minutes at 50° C., 30 minutes at 70° C., and 10 minutes at 25° C.); UNG reaction (1 cycle of 2 minutes at 50° C.); and Taq enzyme activation (1 cycle of 10 minutes at 95° C.), followed by PCR (40 cycles of 15 seconds at 95° C.-1 minute at 60° C.).

After the reaction was completed, the Ct value was calculated using analysis software (Fluidigm Real Time PCR Analysis 4.5.2, available from Fluidigm). The obtained Ct value of each gene was standardized with the Ct value of the internal standard gene GAPDH, and the relative value was calculated when the first group (control group) was set as the standard (1.00).

The results are listed in Table 3.

TABLE 3

| | | First group | Second group |
|---|---|---|---|
| | | (Control group) | (Administration group) |
| Gene | Gene | Dosage of compound (I) (mg/kg) | |
| Symbol | name | 0 | 125 |
| Trim63 | MuRF-1 | 1.00 | 0.81 |
| Fbxo32 | Atrogin-1 | 1.00 | 0.68 |
| Ppargc1a | PGC-1α | 1.00 | 1.28 |
| Slc2a4 | GLUT4 | 1.00 | 1.13 |
| Il6 | IL-6 | 1.00 | 1.10 |

As listed in Table 3, regarding MuRF-1 (Gene Symbol: Trim63) and Atrogin-1 (Gene Symbol: Fbxo32), which are genes encoding ubiquitin ligase that contributes to protein degradation, the gene expression was confirmed to have a tendency to decrease in the second group (administration group) as compared to the first group (control group).

On the other hand, regarding PGC-1α (Gene Symbol: Ppargc1a), GLUT4 (Gene Symbol: Slc2a4), and IL-6 (Gene Symbol: I16), which are genes contributing to muscle increase, the gene expression was confirmed to have a tendency to increase in the second group (administration group) as compared to the first group (control group).

Thus, decrease or increase of the gene expression of these genes by administration of the compound (I) is thought to have contributed to the increase in muscle mass.

### <Testing Example 3> Kinase Activation Analysis Test in Muscle Atrophy Model

For the gastrocnemius muscle extracted in Testing Example 1 above, the phosphorylation state of AMPK and Akt, which are kinases contributing to increase in muscle mass, was evaluated by Luminex measurement and ELISA measurement.

The tissue of the gastrocnemius muscle extracted in Testing Example 1 above was fragmented, and a tissue extract was collected. The total protein concentration was quantified and used for Luminex measurement and ELISA measurement described below so that the total protein amount was 10 μg per well.

For AMPK, PathScan® Total AMPKα Sandwich ELISA Kit (available from Cell Signaling Technology, catalog number: 7961C) was used to measure the total AMPK (t-AMPK), and PathScan® Phospho-AMPKα (Thr172) ELISA Sandwich Kit (available from Cell Signaling Technology, catalog number: 7959C) was used to measure the Thr172 phosphorylated AMPK (p-AMPK).

For Akt, MILLIPLEX® MAP Phospho/Total Akt 2-Plex Magnetic Bead Kit 96-well Plate Assay (available from EMD Millipore, catalog number: 48-618MAG) was used to measure the total Akt amount (t-Akt) and the phosphorylated Akt (p-Akt, Ser473) all at once in one reaction system.

As an internal standard, GAPDH was measured using Total GAPDH Magnetic Bead MAPmate™ (available from EMD Millipore, catalog number: 46-667MAG), and the measured values of the above AMPK and Akt were standardized.

The results are listed in Table 4.

TABLE 4

| | |
|---|---|
| First group | Second group |
| (Control group) | (Administration group) |

Dosage of compound (I) (mg/kg)

| | 0 | 125 |
|---|---|---|
| p-AMPK/t-AMPK | 100.0 | 120.8 |
| p-Akt/t-Akt | 100.0 | 172.9 |

As listed in Table 4, the phosphorylated forms of AMPK and Akt were confirmed to have a tendency to increase in the second group (administration group) as compared to the first group (control group).

Thus, activation of these kinases by administration of the compound (I) is thought to have contributed to the increase in muscle mass.

### Compounding Example 1

Encapsulated formulation having the following composition was produced using an ordinary method. No. 2 hard gelatin capsules were used as the capsules.

«Composition in 1 capsule (1 tablet 200 mg)»

| | |
|---|---|
| Compound (I) | 10.0 mg |
| Cornstarch | 70.0 mg |
| Lactose | 100.0 mg |
| Calcium lactate | 10.0 mg |
| Hydroxypropylcellulose (HPC-L) | 10.0 mg |

### Compounding Example 2

An oral liquid preparation having the following composition was produced using an ordinary method.

«Composition in 1 ampule (100 mL)»

| | |
|---|---|
| Compound (I) | 0.3 mass % |
| Sorvit | 12.0 mass % |
| Sodium benzoate | 0.1 mass % |
| Flavor | 1.0 mass % |
| Calcium sulfate | 0.5 mass % |
| Purified water | the balance (100 mass %) |

1.-8. (canceled)

9. A method for activating AMPK in a patient in need of activating AMPK, comprising steps of:
preparing an AMPK activator containing a compound represented by Formula (I) below as an active ingredient, and
administering an effective amount of the AMPK activator to the patient for activating AMPK.

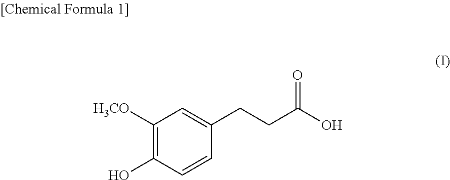 [SMILES: COc1cc(CCC(=O)O)ccc1O]

10. A method for improving motor function in a patient in need of improving motor function, comprising steps of:
preparing a motor function improving agent containing a compound represented by Formula (I) below as an active ingredient, and
administering an effective amount of the motor function improving agent to the patient for improving motor function.

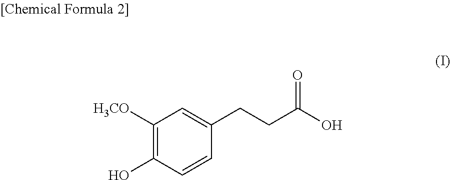 [SMILES: COc1cc(CCC(=O)O)ccc1O]

11. A method for improving muscular endurance in a patient in need of improving muscular endurance, comprising steps of:
preparing a muscular endurance improving agent containing a compound represented by Formula (I) below as an active ingredient, and
administering an effective amount of the muscular endurance improving agent to the patient for improving muscular endurance.

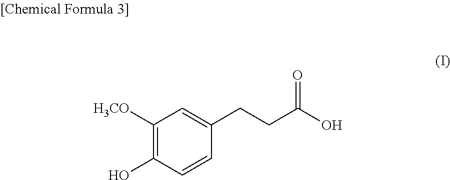 [SMILES: COc1cc(CCC(=O)O)ccc1O]

12. A method for preventing muscle atrophy in a patient in need of preventing muscle atrophy, comprising steps of:
preparing a muscle atrophy preventing agent containing a compound represented by Formula (I) below as an active ingredient, and
administering an effective amount of the muscle atrophy preventing agent to the patient for preventing muscle atrophy.

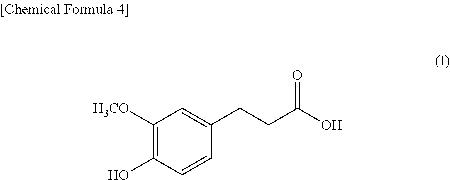 [SMILES: COc1cc(CCC(=O)O)ccc1O]

13. The method for activating AMPK according to claim 9, wherein the AMPK activator is an oral composition.

14. The method for improving motor function according to claim 10, wherein the motor function improving agent is an oral composition.

15. The method for improving muscular endurance according to claim 11, wherein the muscular endurance improving agent is an oral composition.

16. The method for preventing muscle atrophy according to claim 12, wherein the muscle atrophy preventing agent is an oral composition.
